(19) United States
(12) Patent Application Publication
(10) Pub. No.: US 2025/0241548 A1
(43) Pub. Date: Jul. 31, 2025

(54) Synthetic Generation of Face Videos with Plethysmograph Physiology

(71) Applicant: The Regents of the University of California, Oakland, CA (US)

(72) Inventors: Achuta Kadambi, Los Angeles, CA (US); Laleh Jalilian, Santa Monica, CA (US); Zhen Wang, Los Angeles, CA (US); Yunhao Ba, Los Angeles, CA (US); Pradyumna Chari, Los Angeles, CA (US); Oyku Deniz Bozkurt, Los Angeles, CA (US); Maxime Cannesson, Los Angeles, CA (US)

(73) Assignee: The Regents of the University of California, Oakland, CA (US)

(21) Appl. No.: 18/854,940

(22) PCT Filed: Apr. 6, 2023
(86) PCT No.: PCT/US2023/065448

§ 371 Date: Oct. 7, 2024

Related U.S. Application Data
(60) Provisional application No. 63/362,637, filed on Apr. 7, 2022.

(51) Int. Cl. A61B 5/024; A61B 5/0295; G06T 13/40; G06T 17/20

(52) U.S. Cl. CPC A61B 5/02416; A61B 5/0295; G06T 13/40; G06T 17/20; G06T 2210/41

(57) ABSTRACT

Systems and methods for synthetic generation of face videos are described. An embodiment includes receiving an input image; encoding the input image into a UV albedo map, a 3D mesh, an illumination model L_{SH}, and a camera model c; decomposing the UV albedo map into a UV physiological map; varying the UV physiological map according to a target Remote Photoplethysmography (rPPG) signal; generating a plurality of modified PPG UV maps; combining at least one modified PPG UV map with the illumination model L_{SH}, camera model c to render final frames with randomized motion; and generating a synthetic rPPG video using the final frames with randomized motion.

### CROSS-REFERENCE TO RELATED APPLICATIONS

The current application claims priority to U.S. Provisional Patent Application No. 63/362,637, entitled “Synthetic Generation of Face Videos with Plethysmograph Physiology” filed Apr. 7, 2022, the disclosure of which is incorporated by reference herein in its entirety.

### FIELD OF THE INVENTION

The present invention generally relates to synthetic generation of face videos with plethysmography physiology.

### BACKGROUND

Accelerated by telemedicine, advances in Remote Photoplethysmography (rPPG) are beginning to offer a viable path toward non-contact physiological measurement. Unfortunately, the datasets for rPPG are limited as they require videos of the human face paired with ground-truth, synchronized heart rate data from a medical-grade health monitor.

### SUMMARY OF THE INVENTION

Systems and methods of synthetic generation of face videos with plethysmography physiology in accordance with embodiments of the invention are described. An embodiment includes a method of synthetic generation of face videos, including: receiving an input image; encoding the input image into a UV albedo map, a 3D mesh, an illumination model L_{SH}, and a camera model c; decomposing the UV albedo map into a UV physiological map; varying the UV physiological map according to a target Remote Photoplethysmography (rPPG) signal; generating a plurality of modified PPG UV maps; combining at least one modified PPG UV map with the illumination model L_{SH}, camera model c to render final frames with randomized motion; and generating a synthetic rPPG video using the final frames with randomized motion.

In a further embodiment, the at least one modified PPG UV map includes a target pulse signal variation.

In a further embodiment, the camera model c is learned to map a mesh M to image space.

In a further embodiment, the method further includes generating rPPG videos with different attributes including poses, skin tones, and lighting conditions.

In a further embodiment, the UV physiological map is a UV blood map, where the method further comprises first obtaining a spatial concentration of blood f_{blood} of the UV albedo map and then temporally modulate the UV blood map in a way that is consistent with rPPG signals.

In a further embodiment, the method further includes obtaining biophysical parameters directly from the UV albedo map to model underlying blood volume changes.

In a further embodiment, the method further includes training an rPPG network using the generated rPPG videos.

One embodiment includes a system for generating synthetic Remote Photoplethysmography (rPPG) videos, including: at least one processor; and memory coupled to the at least one processor and having programming that causes the processor to execute instructions comprising: receive an input image; encode the input image into a UV albedo map, a 3D mesh, an illumination model L_{SH}, and a camera model c; decompose the UV albedo map into a UV physiological map; vary the UV physiological map according to a target Remote Photoplethysmography (rPPG) signal; generate a plurality of modified PPG UV maps; combine at least one modified PPG UV map with the illumination model L_{SH}, camera model c to render final frames with randomized motion; and generate synthetic rPPG videos using the final frames with randomized motion.

### BRIEF DESCRIPTION OF THE DRAWINGS

FIG. 1 illustrates a system architecture pipeline of a cross-modal synthetic generation model that can generate rPPG face videos given any face image and target rPPG signal as input in accordance with an embodiment of the invention.

FIG. 2 illustrates a scalable system that can generate synthetic rPPG videos with diverse attributes including poses, skin tones and lighting conditions in accordance with an embodiment of the invention.

FIG. 3 illustrates an experimental setup of data collection in accordance with an embodiment of the invention.

FIG. 4 illustrates a table with heart rate estimation results on a real dataset in accordance with an embodiment of the invention.

FIG. 5 illustrates, left, ablation study with a model pre-trained with synthetic dataset outperforms model pre-trained on either light or dark skin tones alone, and right, bias mitigation, where the standard deviation of MAE and RMSE of a deep rPPG models trained with real and synthetic dataset are smaller than real data alone and the traditional models in accordance with an embodiment of the invention.

FIG. 6 illustrates an example that shows that PRN trained with synthetic data (above) generalizes better than PRN trained with real data (bottom) on UBFC-rPPG dataset in accordance with an embodiment of the invention.

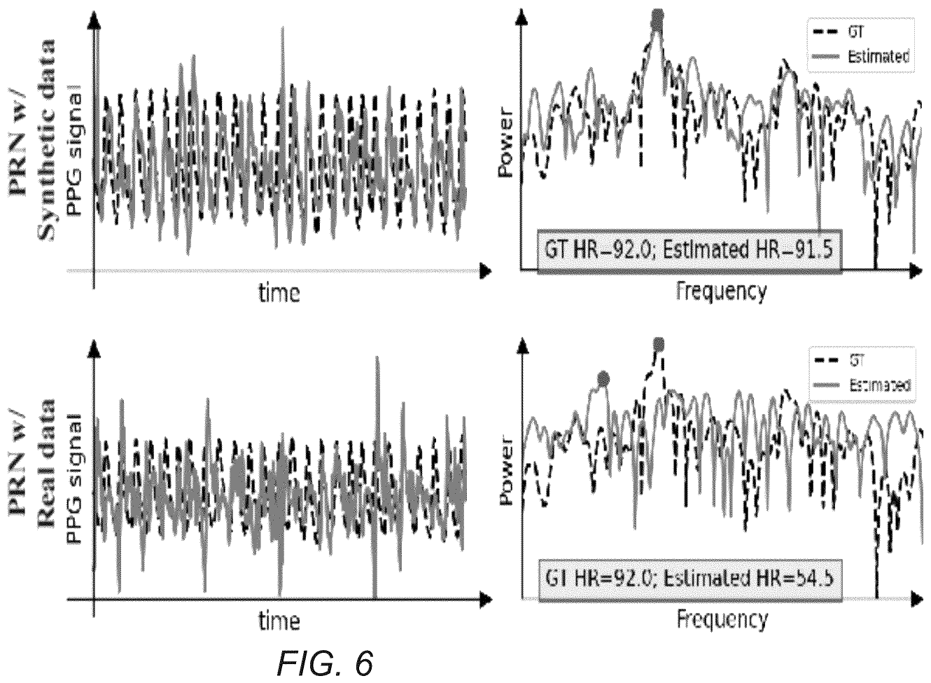

FIG. 7 illustrates example frames of generated synthetic video with incorporated PPG signals into a reference image in accordance with an embodiment of the invention.

FIG. 8 illustrates a computer system architecture for synthetic generation of rPPG videos in accordance with an embodiment of the invention.

### DETAILED DESCRIPTION OF THE DRAWINGS

Systems and methods in accordance with various embodiments of the invention provide for scalable biophysics-based learning models that can render realistic remote Photoplethysmography (rPPG) videos with high fidelity to underlying Blood Volume Pulse (BVP). In many embodiments, the synthetically generated videos can be directly utilized to improve the performance of deep rPPG methods. In many embodiments of the system, a rendering model can be deployed to generate data for underrepresented groups, which can provide an effective method to further mitigate demographic bias in rPPG frameworks. Moreover, to facilitate the rPPG advancement, systems in accordance with many embodiments can use real rPPG datasets that includes diverse skin tones. The dataset can be used to benchmark performance across different demographic groups in this area.

Systems in accordance with many embodiments are capable of generating physio-realistic synthetic rPPG video sequences given a reference image and a target rPPG signal as input. The generated videos can be of high fidelity to underlying BVP variations as specified by the input rPPG waveform. Systems in accordance with many embodiments can generate and use a biophysically interpretable manipulation of a UV albedo map obtained from 3D Morphable Face Model (3DMM) and can enable rendering rPPG videos with large variations of various attributes such as facial appearance and expression, head motions and environmental lighting, among others.

Systems in accordance with many embodiments can include receiving an input image, encoding the input image into a UV albedo map, a 3D mesh, an illumination model L_{SH}, and a camera model c, decomposing the UV albedo map into a UV physiological map such as a UV blood map among others, varying the UV blood map according to a target Remote Photoplethysmography (rPPG) signal, generating several modified PPG UV maps, combining at least one modified PPG UV map with the illumination model L_{SH}, camera model c to render final frames with randomized motion.

In many embodiments, a system can use a large collection of in-the-wild images and PPG recordings, allowing an easy and scalable mechanism to ensure a balanced presence of demographics in generated videos. Systems in accordance with many embodiments, in terms of computational complexity, can achieve up to 1000 times faster than currently available systems.

As noted, accelerated by telemedicine, advances in Remote Photoplethysmography (rPPG) are beginning to offer a viable path toward non-contact physiological measurement. Unfortunately, the datasets for rPPG are limited as they can require videos of the human face paired with ground-truth, synchronized heart rate data from a medical-grade health monitor. Also troubling is that the datasets are not inclusive of diverse populations, e.g., current real rPPG facial video datasets are imbalanced in terms of races or skin tones, leading to accuracy disparities on different demographic groups. Accordingly, systems in accordance with many embodiments provide a scalable biophysical learning based processes to generate physio-realistic synthetic rPPG videos given a reference image and target rPPG signal. Systems in accordance with many embodiments show further improvments in physiological measurement and reduce bias among different groups. Many embodiments of the systems can collect a large rPPG dataset of its kind with a diverse presence of subject skin tones, and this can serve as a benchmark dataset for different skin tones in this area and ensure that advances of the technique can benefit all people for healthcare equity.

Photoplethysmography (PPG) is an optical technique that measures vital signs such as Blood Volume Pulse (BVP) by detecting the light reflected or transmitted through the skin. Remote Photoplethysmography (rPPG) based on camera videos can have several advantages over the conventional PPG methods. It is non-contact thus allowing for a wide range of applications in e.g. neonatal monitoring. It causes no skin irratation and prevents the risk of developing into infection for those whose skins are fragile and sensitive to the adhesive sensing electrodes. As cameras are ubiquitous in electronic device nowadays (such as smartphones, laptops), rPPG can be applied for telemedicine with patients at home and no equipment set-up is needed. Camera-based rPPG techniques have also been used in other applications such as driver monitoring and face anti-spoofing.

Traditional rPPG methods either use Blind Source Separation (BSS) or models based on skin reflectance to separate out the pulse signal from the color changes on the face. These methods usually require pre-processing such as face tracking, registration and skin segmentation. More recently, deep learning and convolutional neural networks (CNN) have been more popular due to its expressiveness and flexibility. CNNs learn the mapping between the pulse signal and the color variations with end-to-end supervised training on the labeled dataset, thus achieving state-of-the-art performance on the vital sign detection. However, the performance of data-driven rPPG networks can hinge on the quality of the dataset.

There are some efforts on collecting a large rPPG dataset for better physiological measurement. Nonetheless, there exists several practical constraints towards collecting real patient data for medical purposes. These include: (1) demographic biases (such as race biases) in society that translate to data. A diverse rPPG dataset may not be accessible for some countries/regions due to geographical distribution of skin colors as reflected in their skin tone world map for indigenous people. (2) necessity of intrusive/semi-intrusive traditional methods for collection of data, (3) patient privacy concerns, and (4) requirement of medical-grade sensors to generate the data. Hence, there is a pressing need for the concept of ‘digital patients’: physiologically accurate graphical renders that may assist development of algorithms and techniques for improvement of diagnostics and healthcare. Accordingly, systems in accordance with many embodiments provide a neural rendering instantiation in the rPPG field.

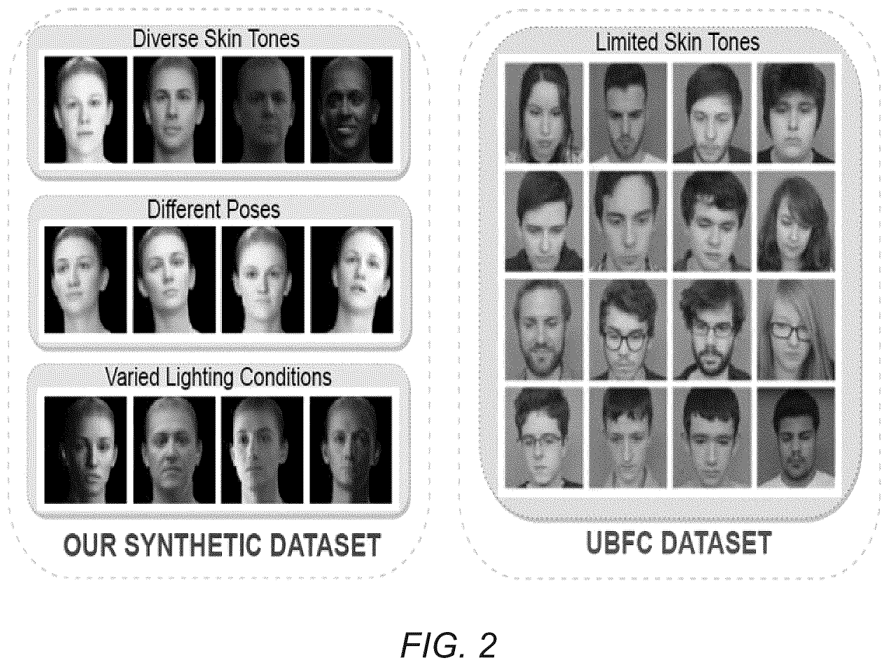

For decades, computer graphics has been a driving force for the visuals seen in movies and games. Systems in accordance with many embodiments can harness computer graphics techniques to create not just photorealistic humans, but physio-realistic humans. Many embodiments combine modalities of image and waveform to learn to generate a realistic video that can reflect underlying BVP variations as specified by the input waveform. Systems in accordance with several embodiments achieve this by an interpretable manipulation of UV albedo map obtained from the 3D Morphable Face Model (3DMM). Systems in accordance with many embodiments can provide a model that can generate rPPG videos with large variation of various attributes such as facial appearance and expression, head motions and environmental lighting, among others. FIG. 2 illustrates a scalable system that can generate synthetic rPPG videos with diverse attributes such as poses, ski tones and lighting conditions in accordance with an embodiment of the invention.

Systems in accordance with many embodiments provide a scalable physics-based learning model that can render realistic rPPG videos with high fidelity with respect to underlying blood volume variations. The synthetically generated videos can be directly utilized to improve the performance of the state-of-the-art deep rPPG methods. Notably, the corresponding rendering model can also be deployed to generate data for underrepresented groups, which provides an effective method to further mitigate the demographic bias in rPPG frameworks.

To facilitate the rPPG research, systems in accordance with many embodiments can use a real rPPG dataset that includes diverse skin tones. The dataset can be used to benchmark performance across different demographic groups in this area.

### Pipeline Cross-Modal Synthetic Generation Model Pipeline

Systems in accordance with many embodiments, an input image can be encoded into UV albedo map, 3D mesh, illumination model L_{SH} and camera model c. The UV albedo map can be decomposed into blood map, and the system can vary the UV blood map according to the target rPPG signal and generate the modified PPG UV maps. The modified PPG UV map that includes the target pulse signal variation can be combined with L_{SH}, c to render the final frames with randomized motion.

rPPG Methods:

rPPG techniques can aim to recover the blood volume change in the skin that is synchronous with the heart rate from the subtle color variations captured by a camera. Signal decomposition methods can include that utilizes Principal Component Analysis (PCA) on the raw traces and chooses the decomposed signal with the largest variance as the pulse signals and Independent Component Analysis (ICA) that demixes the raw signals and determines the separated signals with largest periodicity as the pulse. PCA and ICA can be purely statistical approaches that do not use any prior information unique to rPPG problems. A chrominance-based method (CHROM) can be uswed to extract the blood volume pulse by assuming a standardized skin-color to white-balance the image and then linearly combine the chrominance signals. Plane Orthogonal to Skin-tone (POS) projects the temporally normalized raw traces onto a plane that is orthogonal to the light intensity change, thus canceling out the effect of that. CNNs have achieved state-of-the-art results on vital sign detection due to their flexibility. The representation for rPPG estimation can be efficiently learned in an end-to-end manner with the annotated datasets instead of handcrafted features for traditional methods. Many embodiments of the system can use two representative work, PhysNet and PRN, to demonstrate the performance of the rPPG models on both real and synthetic datasets.

Real rPPG Datasets:

There have been efforts on collecting real datasets for more accurate physiological sensing. However, these datasets are usually very limited in the number of subject participants and also biased towards certain demographic group. Some work includes subject with darker skin types, but the number is still very limited. Making machine learning methods equitable is of increasing interest in medical domain. There is a lack of a benchmark dataset to measure the performance of various rPPG methods on diverse skin tones, especially dark skin tones in rPPG area. Some prior techniques proposed a dataset that only contains dark skin tones. However, the actual videos are not shared but the color space values of skin region of interest. The current best-performing deep learning algorithms require sizeable input data. The rPPG model trained on such a biased dataset may easily disadvantage certain underrepresented groups in the dataset. The lack of such a benchmark dataset to systematically and rigorously evaluate various methods on diverse skin tones makes it hard to ensure that the rPPG methods deployed into the society would not cause biases against certain groups that are underrepresented. Many embodiments can include a real dataset that represents a first step towards filling this gap.

Synthetic Generation of rPPG Videos:

The real rPPG dataset construction is a laborious process and generally takes a large amount of time for collection and administrative work for Institutional Review Board (IRB) approval. Therefore, it is beneficial to have a scalable method that can generate large-scale synthetic rPPG datasets for data augmentation. Accordingly, systems in accordance with many embodiments provide for synthetic generation processes that can generate diverse appearance with any in-the-wild image and target rPPG signal as input and the generation can be a forward pass of a neural network. Systems in accordance with many embodiments provide a scalable model and processes that can generate synthetic datasets with a given reference image and target rPPG signal. The generated videos can be used to train the state-of-the-art rPPG networks.

### Synthesizing Biorealistic Face Videos

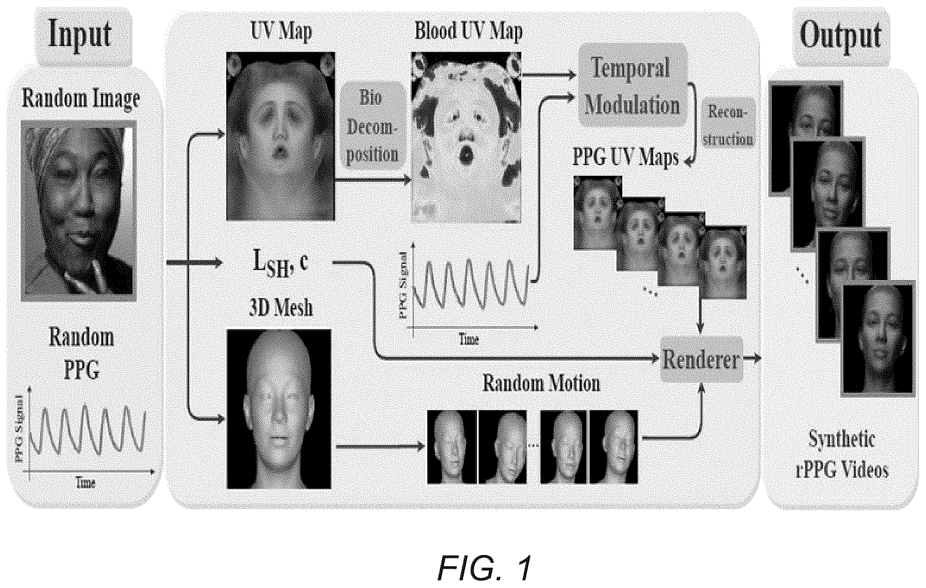

In many embodiments, a 3DMM model can be used to obtain the facial albedo maps and then obtain facial blood maps from the extracted albedo by analyzing light transport in the skin. Further details about how to generate synthetic facial videos with the decomposed blood maps and the source of the input facial images and PPG waveforms are described herein. FIG. 1 illustrates a synthetic generation pipeline in accordance with an embodiment of the invention.

### Non-Linear 3DMM

To generate faces with different poses, illuminations and desirable rPPG signal variations, many embodiments of the system can infer the 3D shape and albedo parameters of the face. In many embodiments, a system can use DECA to predict subject-specific albedo, shape, pose, and lighting parameters from an image. A system may use a statistical 3D head model FLAME to output a mesh M with a number of verticies (e.g., n=5023 vertices). A camera model c can be learned to map the mesh M to image space. Since there may be no appearance model in FLAME, the linear albedo subspace of Basel Face Model (BFM) can be used and the UV layout of BFM can be converted to be compatible with FLAME. Systems in accordance with many embodiments can output a UV albedo map A with a learnable coefficient α. By expressing illumination model as the Spherical Harmonics (SH), the shaded face image can be represented as the following equation:

$${B ( α , l , {N}_{uv} )}_{i , j} = {A ( α )}_{i , j} ⊙ {\sum }_{k = 1}^{9} {l}_{k} {H}_{k} ( {N}_{i , j} ) , \qquad ( 1 )$$

where Hₖ is the SH basis, Iₖ are the corresponding coefficients and ⊙ denotes the Hadamard product. N_{i,j} is the normal map expressed in the UV form. The final texture image can be obtained by rendering the image using the mesh M, shaded image B, and the camera model c through a rendering function [symbol] (·):

$${I}_{r} = ℛ ( M , B , c ) . \qquad ( 2 )$$

As rPPG can be essentially the change of blood volume in the face, systems in accordance with many embodiments can first obtain the spatial concentration of blood f_{blood} of the UV albedo A and then temporally modulate the UV blood albedo map in a way that is consistent with the rPPG signals. Descirbed below are how this biophysically interpretable manipulation can be achieved.

### Light Transport in the Skin:

In order to obtain blood map f_{blood} on the face, systems in accordance with many embodiments can analyze light transport in the skin to build the connection between face albedo and f_{blood}. Following a spectral image formation model, the original UV face albedo A_{c} with c∈{R, G, B} can be reconstructed by integrating the product of the camera spectral sensitivities S_{c}, the spectral reflectance R, and the spectral power distribution of the illuminant E over wavelength λ:

$${A}_{c} = {\int }_{λ} E ( λ ) R ( {f}_{mel} , {f}_{b l o o d} , λ ) {S}_{c} ( λ ) d λ . \qquad ( 3 )$$

An optical skin reflectance model with hemoglobin f_{blood} and melanin map fₘₑₗ as parameters can be utilized to define the wavelength-dependent skin reflectance R(fₘₑₗ, f_{blood}, λ). Specifically, systems in accordance with many embodiments can apply a two-layer skin model that characterizes the transmission through the epidermis T_{epidermis} and reflection from the dermis R_{dermis}:

$$R ( {f}_{mel} , {f}_{b l ood} , λ ) = {{T}_{epidermis} ( {f}_{mel} , λ )}^{2} {R}_{d e r m i s} ( {f}_{b l ood} , λ ) . \qquad ( 4 )$$

The transmittance in epidermis can be modeled by Lambert-Beer law as light not absorbed by the melanin in this layer is propagated to the dermis:

$${T}_{epidermis} ( {f}_{mel} , λ ) = {e}^{- {μ}_{a . epidermi s ( {f}_{mel} , λ )}} , \qquad ( 5 )$$

where μ_{a.epidermis}(fₘₑₗ, λ) is the absorption coefficient of the epidermis. More specifically,

$${μ}_{a . e p i d e r m i s} ( {f}_{mel} , λ ) = {f}_{mel} {μ}_{a . mel} ( λ ) + ( 1 - {f}_{mel} ) {μ}_{skin baseline} ( λ ) , \qquad ( 6 )$$

where μ_{a.mel} is the absorption coefficient of melanin and μ_{skinbaseline} is baseline skin absorption coefficient.

The reflectance in dermis can be modeled using the Kubelka-Munk theory, and the proportion of light remitted from a layer is given by:

$${R}_{demis} ( {f}_{b l ood} , λ ) = \frac{( 1 - {β}^{2} ) ( {e}^{K {d}_{p d}} - {e}^{- K {d}_{p d}} )}{( 1 + {β}^{2} ) {e}^{K {d}_{p d}} - {( 1 - β )}^{2} {e}^{- K {d}_{p d}}} , \qquad ( 7 )$$

where d_{pd} is the thickness of the dermis, and K and β are related to the absorption of the medium contained within the dermis (e.g. blood). For simplicity of notation, the dependence of K and β on f_{blood} and λ can be dropped in Equation (7).

### Biophysical Decomposition and Variation of UV Albedo Map:

With the light transport theory of the skin, systems in accordance with many embodiments can apply a physics-based learning framework (e.g., BioFaceNet) to obtain f_{blood} from albedo A. The wavelengths can be discretized into a number of parts and spacing (e.g., 33 parts from 400 nm to 720 nm with 10 nm equal spacing). Many embodiments of the system can use an autoencoder architecture and use a fully-convolutional network as encoder to predict the hemoglobin and melanin maps and fully-connected networks to encode the parameters for lighting E and camera spectral sensitivities S_{c}. The model-based decoder is then to reconstruct the albedo with all the learned parameters according to Equation (3).

Systems in accordance with many embodiments can obtain biophysical parameters directly from the UV albedo maps instead of the facial images. This arrangement may allow to model the underlying blood volume changes more precisely regardless of the environmental illumination variations. Systems in accordance with many embodiments, a model can be trained to minimize the following loss function:

$$ℒ = {w}_{1} {ℒ}_{appearance} + {w}_{2} {ℒ}_{C a m e r a P r ior} , \qquad ( 8 )$$

where the appearance loss [symbol] _{appearance} is the L2 distance between the reconstructed UV map A_{linRecon} and the original one in the linear RGB space A_{linRGB}-Many embodiments can convert A to linear space by inverting the Gamma transformation with γ=2.2. To make the problem more constrained, many embodiments introduce the additional camera prior loss: [symbol] _{CameraPrior}=∥b∥₂², where b is the prior for the camera spectral sensitivities. w₁ and w₂ are the weights for the reconstructed loss and camera prior loss, respectively.

To reflect the change of the target rPPG signal on the face, many embodiments temporally vary the UV blood map f_{blood} linearly with the target rPPG signal in the test phase. Given the blood map of a reference UV map (e.g. the UV blood map of first frame), many embodiments generate the UV blood map of the consequent frames as the multiplication of the UV blood map of the reference frame and a ratio scalar that is calculated as the ratio of pₜ (rPPG signal at time t) and p_{ref} (rPPG signal at the reference time). Then the modified UV blood map of each frame that contains the desired rPPG signal can be reconstructed using the BioFaceNet decoder to get UV map. The final image can be rendered using the UV map combined with illumination and camera model according to Equation (2).

For the purpose of simulating real-world scenarios where the subject might move in the collection process, many embodiments can randomize the poses in the generation of the sequence of the frames by adding a small random value to the pose and expression parameter of the previous frame.

### Face Image Dataset:

To generate synthetic rPPG videos with diverse face appearances, systems in accordance with many embodiments can use the public in-the-wild face datasets BUPT-Balancedface. It can be categorized according to ethnicity (e.g. Caucasian, Indian, Asian and African). In many embodiments, these images can be used as the reference images for generating the synthetic videos as shown in FIG. 1 in accordance with an embodiment of the invention. Although FIG. 1 illustrates a particular system pipeline of a cross-modal synthetic generation model that generates rPPG videos, any of a vareity of system configurations can be utilized as appropriate to the requirements of specific applications in accordance with embodiments of the invention.

### Ppg Recordings:

To synthesize videos of a given input PPG signal, systems in accordance with many embodiments may use PPG waveforms recordings from BIDMC PPG and Respiration Dataset. It can include a number of contact PPG recordings of a certain length (e.g., 53 videos of 8 minutes in length) with sampling frequency 125 Hz. Many embodiments sample it correspondingly with the video frame rate (e.g., 30 Hz) and the first sequences of time length L can be used where L is the duration of the generated video.

Many embodiments can use two state-of-the-art deep rPPG networks PhysNet and PRN to benchmark the performance on both real and synthetic datasets. PhysNet and PRN both utilize 3D convolutional neural networks (3D-CNN) architecture to learn spatio-temporal representation of the rPPG videos and predict the rPPG signal in the facial videos. PRN differs in that it uses residual connection for convolutional layers. They take consecutive frames of length T as the input, and its output is the corresponding BVP value for each input frame. The Negative Pearson loss is used to measure the difference between the ground-truth PPG signal p and the estimated rPPG signal p:

$${L}_{ppg} ( p , \hat{p} ) = 1 - \frac{T {\sum }_{i} {p}_{i} {\hat{p}}_{i} - {\sum }_{i} {p}_{i} {\sum }_{i} {\hat{p}}_{i}}{\sqrt{( T {\sum }_{i} {p}_{i}^{2} - {( {\sum }_{i} {p}_{i} )}^{2} ) ( T {\sum }_{i} {\hat{p}}_{i}^{2} - {( {\sum }_{i} {\hat{p}}_{i} )}^{2} )}} , \qquad ( 9 )$$

where all the summation is over the length of frames T.

### Implementations:

For the training of BioFaceNet, systems in accordance with many embodiments can use a number (e.g., 3000) of face albedo images with a number (e.g., 750) images in each race. Many embodiments can use a percentage of the (e.g., 80%) images for training and a percentage (e.g., 20%) for validation. The weight w₁ and w₂ for the loss can be 1e⁻³ and 1e⁻⁴ respectively. The learning rate can be set as 1e⁻⁴ and the number of epochs can be e.g., 200. For the generation of synthetic videos, many embodiments can set the length of generated frames L as e.g., 2100.

The bounding boxes of the videos can be generated using a pretrained Haar cascade face detection model. For each video, one bounding box can be detected and increased a certain percentage (e.g., 60%) in each direction before the frames are cropped. To be consistent with the original papers, each frame can be resized to a number of pixels (e.g., 128×128 pixels) using bilinear interpolation for PhysNet and e.g., 80×80 for PRN. The length of training clips T is e.g., 128 for PhysNet and e.g., 256 for PRN. The Adam optimizer can be used and the learning rate is set as 1e⁻⁴.

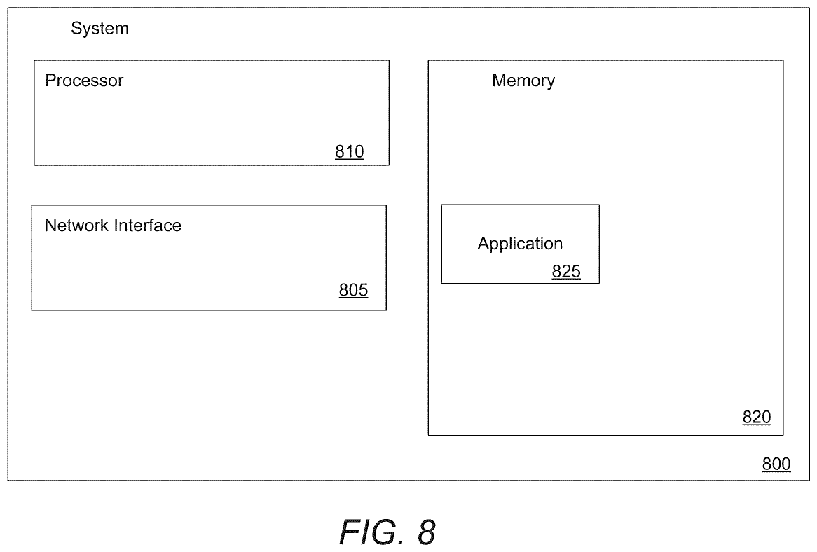

A computer system architecture for synthetic generation of rPPG videos in accordance with many embodiments of the invention is illustrated in FIG. 8. The system 800 system includes a processor 810 that can be configured to execute instructions, a network interface that can communicate with one or more external interfaces, and a memory 820 that can include one or more applications 825. Although FIG. 8 illustrates a particular computer system architecture that can generate synthetic rPPG videos, any of a variety of computer architectures can be utilized as appropriate to the requirements of specific applications in accordance with embodiments of the invention.

### Experiments

Described now are datasets used for the experiments and evaluation protocol.

### UCLA-rPPG Real Dataset:

In order to benchmark the performance of current rPPG estimation methods, we collect a real dataset of 104 subjects. The setting is faulty for two of them so we dropped their samples. Finally, the dataset consists of 102 subjects of various skin tone, age, gender, ethnicity and race. The Fitzpatrick (FP) skin type scale of the subjects varies from 1-6. For each subject, we record 5 videos of about 1 minute each (1790 frames at 30 fps). After removing erroneous videos we have total 503 videos. All the videos in our dataset are uncompressed and synchronized with the ground truth heart rate.

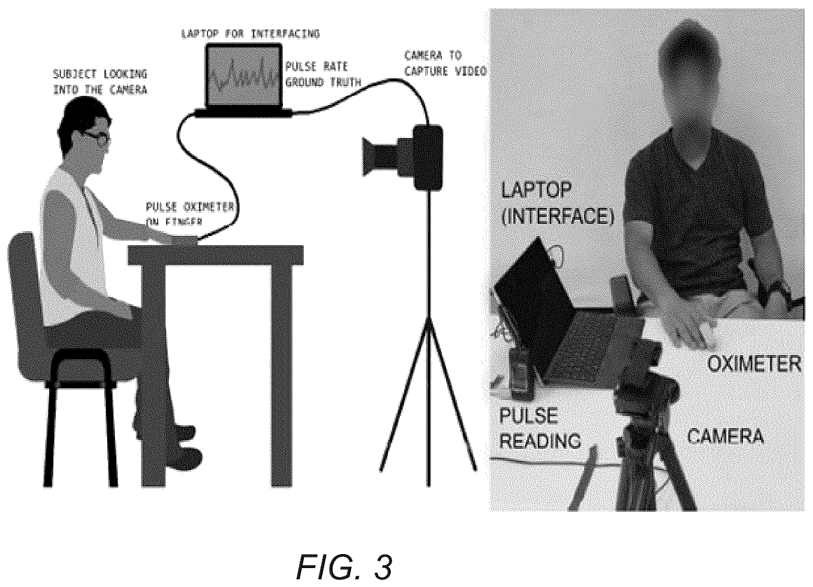

FIG. 3 illustrates a data collection process of a real dataset UCLA-rPPG in accordance with an embodient of the invention. The left part of the figure is a cartoon illustration of the data collection process. The right part of the figure is a photo depicting the actual data collection process. The human subjects wear an oximeter on finger and looks into the camera. Both the camera and the oximeter are connected to a laptop to get synchronous data.

### UBFC-rPPG:

UBFC-rPPG database contains 42 front facing videos of 42 subjects and corresponding ground truth PPG data recorded from a pulse oximeter. The videos are recorded at 30 frames per second with a resolution of 640×480. Each video is roughly one minute long.

### Metrics:

To evaluate how the heart rate estimates compare with gold-standard heart rates obtained from gold-standard pulse waves, we use the following four metrics Mean absolute error (MAE), Root Mean Squared Error (RMSE), Pearson's Correlation Coefficient (PCC) and Signal-to-Noise Ratio (SNR).

For traditional baseline methods POS, CHROM and ICA we compare, we use iPhys toolbox to get the estimated rPPG waveforms. The output rPPG signals are normalized by subtracting the mean and dividing by the standard deviation. We filter all the model outputs using a 6th-order Butterworth filter with cut-off frequencies 0.7 and 2.5 Hz. The filtered signals are divided into 30-second windows with 1-second stride and the above four evaluation metrics are calculated on these windows and averaged.

### Performance on UCLA-rPPG

For the study of this work, we split the subjects into three skin tone groups based on the Fitzpatrick skin type. They are light skin tones, consisting of skin tones in the FP 1 and 2 scales, medium skin tones, consisting of skin tones in the FP 3 and 4 scales, and dark skin tones, consisting of skin tones in the FP 5 and 6 scales. This aggregation helps compare experimental results on skin tones more objectively. Since our ultimate goal is to improve the performance on our dataset, we first train on all the synthetic data and then finetune on the real data for the models trained with both real and synthetic data. For training and testing deep rPPG networks PhysNet and PRN on real dataset, we randomly split all the subjects into training, validation and test set with 50%, 10% and 40% and all the test results are averaged on three random splits. The validation set is used to select the best epoch for testing the model.

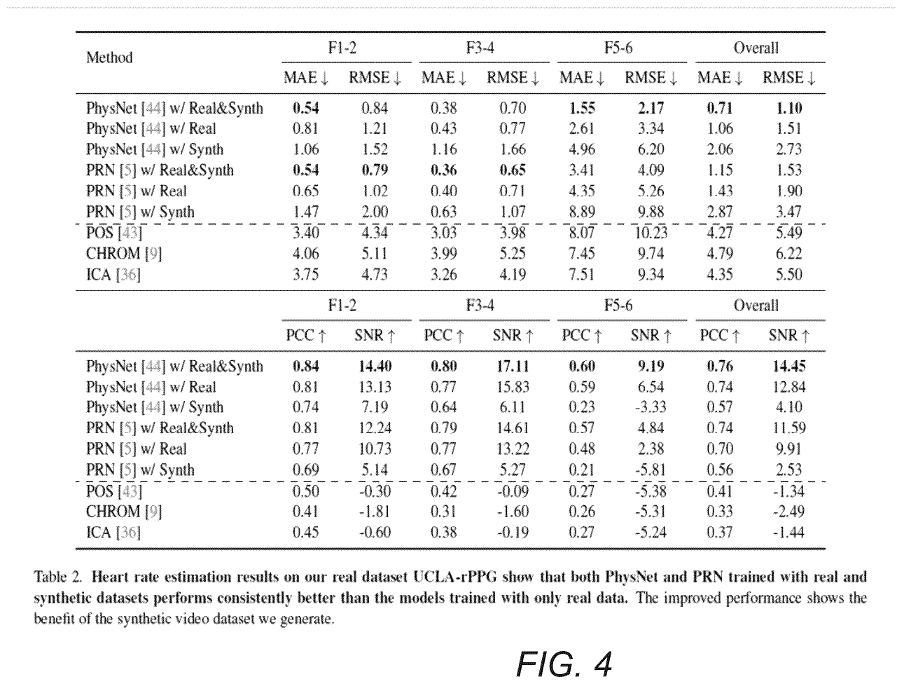

FIG. 4 illustrates (Left Ablation study) a model pre-trained with all synthetic dataset outperforms these pre-trained on either light or dark skin tones alone; Right: Bias mitigation, the standard deviation of MAE and RMSE of the deep rPPG models trained with real and synthetic dataset are smaller than real data alone and the traditional models.

We report results on the three groups and overall performance using evaluation metrics of MAE, RMSE, PCC and SNR in table: vital. In general, models trained with both real and synthetic data perform consistently better than using real data alone on all the skin tones for all evaluation metrics. PhysNet trained with both real and synthetic data achieved the best overall MAE result 0.71 BPM, with 33% reduction in error compared with PhysNet trained with only real data (1.06 BPM). Notably, the performance improvement is most significant on dark skin stones F5-6 group with 41% and 35% reduction in MAE and RMSE respectively for PhysNet. The same phenomenon is also observed for PRN, where the improvement is most noticeable for darker skin tones. We attribute this to the introduction of synthetic videos generated. The other two metrics PCC and SNR also validate the superiority of the model trained with both real and synthetic datasets. The results for traditional methods POS, CHROM and ICA are far worse than the deep learning methods, as these methods usually takes the average of all the pixels and ignore the inhomogeneous spatial contribution of the pixels to pulsatile signals.

### Bias Mitigation:

To evaluate the bias of various rPPG methods on subjects with diverse skin tones, we use the standard deviation of the MAE and RMSE results on three skin tone groups. From the right of fig: bias_plot, we can see the standard deviation of PhysNet with both real and synthetic dataset is the smallest and the MAE disparity among all the three groups are reduced by 45% (from 0.95 BPM to 0.52 BPM) compared with the model trained with only real dataset. Similarly, the standard deviations of both metrics MAE and RMSE for PRN are also reduced for the model trained with both real and synthetic datasets.

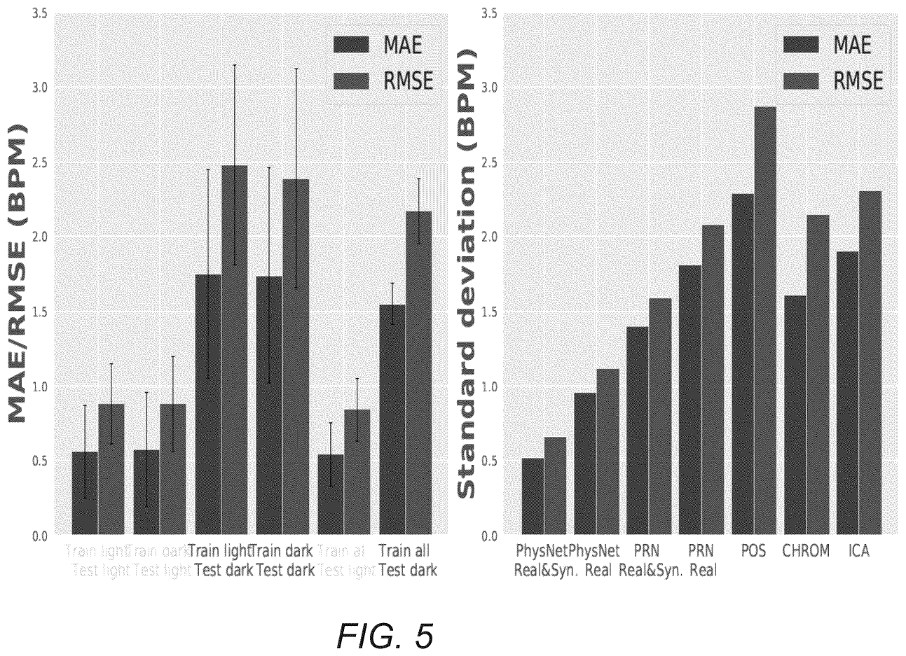

FIG. 5 illustrates an example shows that PRN trained with synthetic data (above) generalizes better than PRN trained with real data (bottom) on UBFC-rPPG dataset. The waves are more aligned with the ground-truth PPG wave (dashed black line) and the power spectrum plot is also more consistent with the ground-truth for the PRN trained with synthetic data.

### Ablation Study:

We first pre-train the PhysNet with either light skin tones (subjects with race Caucasian in the synthetic dataset) or dark skin tones (subjects with race African), then finetune the model on real dataset and test the model on real subjects with either light skin tones or dark skin tones. From the left of FIG. 5, we can see the model with the pre-trained rPPG network on diverse races are consistently better than these on a single race. The improvement is more obvious on dark skin tones test set. This demonstrates the benefits of a diverse synthetic dataset.

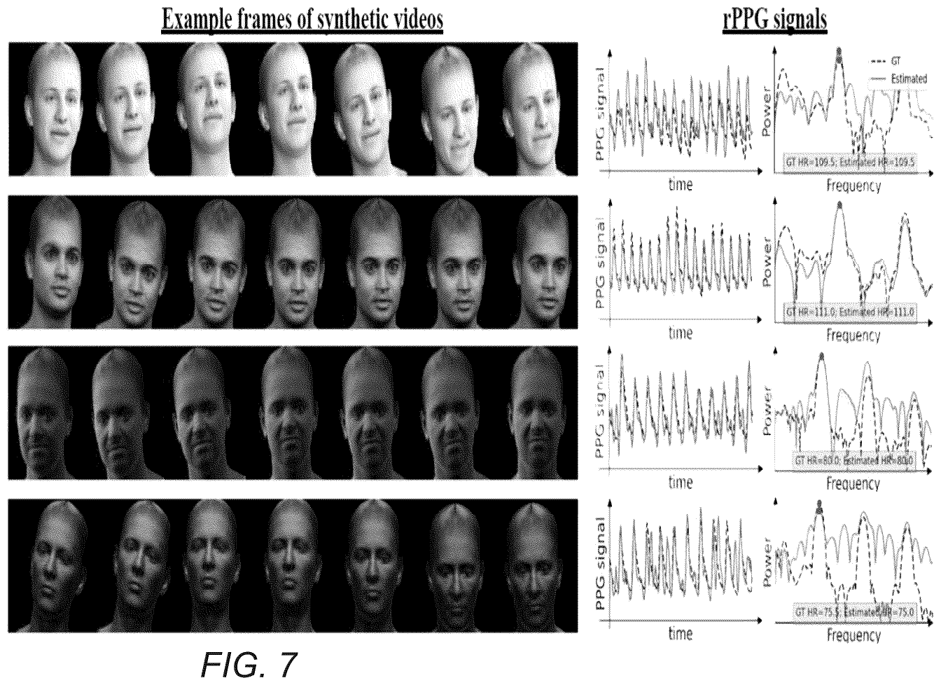

FIG. 7 illustrates example frames of generated synthetic videos in accordance with an embodiment of the invention. A framework in accordance with many embodiments has successfully incorporated PPG signals into the reference image. The estimated pulse waves from PRN for generated synthetic videos are highly correlated to the ground-truth waves, and the heart rates are preserved as shown in the power spectrum plot.

### Performance on UBFC-rPPG

We use the model with best performance on our real dataset to test them on UBFC-rPPG dataset along with the traditional methods. Since this is a cross-dataset evaluation for the model trained on UCLA-rPPG, we test the deep learning models on all the subjects in UBFC-rPPG. All the results with four evaluation metrics are reported in tab:ubfc. While the synthetic dataset performs worse than the models trained in our real dataset, the performance gain is more obvious in UBFC dataset. The MAE of PhysNet trained on synthetic dataset achieved the lowest MAE and RMSE (0.84 BPM and 1.76 BPM respectively). The explanation for this observation is that when the distribution of the dataset is similar to the distribution of the test data as in the intra-dataset setting in our real dataset, the benefits of synthetic datasets are not straightforward. The models trained on real dataset perform worse on generalizing to another dataset due to different environmental setting such as lighting. We also give a qualitative study in FIG. 7 that shows that the rPPG wave extracted using our synthetic dataset resemble more closely to the ground-truth than that using real dataset. As a result, it gives more accurate heart rate estimation.

### Visualization

As shown in FIG. 7 in accordance with an embodiment, a system can successfully produce synthetic avatar videos that reflect the associated underlying blood volume changes. Estimated pulse waves from the synthetic videos are closely aligned with the ground truth. The power spectrum of the PPG waves with a clear peak near the gold-standard HR value also validates the effectiveness of the incorporation of pulsatile signals.

### Limitations:

Though our synthetic dataset could be used to achieve state-of-the-art results (on UBFC-rPPG datasets, it alone can generalize even better than the model trained on real dataset) for heart rate estimation, the facial appearance is not photo-realistic, which may still degrade the performance due to sim2real gap. We are not focused on modeling the background in the generated videos in this work. However, it is found in that the background can be utilized for better pulsatile signals extraction. Also we vary the UV blood map linearly according to the target rPPG signals in the synthetic generation method. While this yields reasonable empirical results, we believe biophysical model based manipulation of the UV blood map could further improve the performance of the synthetic generation.

Although specific implementations for synthetic generation of face videos with plethysmography physiology are discussed above with respect to FIG. 1, any of a variety of implementations utilizing the above discussed techniques can be utilized for synthetic generation of face videos with plethysmography physiology in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. A method of synthetic generation of face videos, comprising:
receiving an input image;
encoding the input image into a UV albedo map, a 3D mesh, an illumination model L_{SH}, and a camera model c;
decomposing the UV albedo map into a UV physiological map;
varying the UV physiological map according to a target Remote Photoplethysmography (rPPG) signal;
generating a plurality of modified PPG UV maps;
combining at least one modified PPG UV map with the illumination model L_{SH}, camera model c to render final frames with randomized motion; and
generating a synthetic rPPG video using the final frames with randomized motion.

2. The method of claim 1, wherein the at least one modified PPG UV map includes a target pulse signal variation.

3. The method of claim 1, wherein the camera model c is learned to map a mesh M to image space.

4. The method of claim 1, further comprising generating rPPG videos with different attributes including poses, skin tones, and lighting conditions.

5. The method of claim 1, wherein the UV physiological map is a UV blood map, where the method further comprises first obtaining a spatial concentration of blood f_{blood} of the UV albedo map and then temporally modulate the UV blood map in a way that is consistent with rPPG signals.

6. The method of claim 1, further comprising obtaining biophysical parameters directly from the UV albedo map to model underlying blood volume changes.

7. The method of claim 1, further comprising training an rPPG network using the generated rPPG videos.

8. A system for generating synthetic Remote Photoplethysmography (rPPG) videos, comprising:
at least one processor; and
memory coupled to the at least one processor and having programming that causes the processor to execute instructions comprising:
receive an input image;
encode the input image into a UV albedo map, a 3D mesh, an illumination model L_{SH}, and a camera model c;
decompose the UV albedo map into a UV physiological map;
vary the UV physiological map according to a target Remote Photoplethysmography (rPPG) signal;
generate a plurality of modified PPG UV maps;
combine at least one modified PPG UV map with the illumination model L_{SH}, camera model c to render final frames with randomized motion; and
generate synthetic rPPG videos using the final frames with randomized motion.

9. The system of claim 8, wherein the at least one modified PPG UV map includes a target pulse signal variation.

10. The system of claim 8, wherein the camera model c is learned to map a mesh M to image space.

11. The system of claim 8, wherein the processor further executes instructions comprising generating rPPG videos with different attributes including poses, skin tones, and lighting conditions.

12. The system of claim 8, wherein the UV physiological map is a UV blood map, wherein the processor further executes instructions comprising first obtaining a spatial concentration of blood f_{blood} of the UV albedo map and then temporally modulate the UV blood map in a way that is consistent with rPPG signals.

13. The system of claim 8, wherein the processor further executes instructions comprising obtaining biophysical parameters directly from the UV albedo map to model underlying blood volume changes.

14. The system of claim 8, wherein the processor further executes instructions comprising training an rPPG network using the generated rPPG videos.
